(19) United States
(12) Patent Application Publication
(10) Pub. No.: US 2008/0081827 A1
(43) Pub. Date: Apr. 3, 2008

(54) Use of a Pyrazole-Derived Compound that is an Antagonist for Cannabinoid CB1 Receptors, for Treating or Preventing Chronic Bronchitis or Chronic Obstructive Bronchopneumopathy

(75) Inventor: Marie Sebille, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI AVENTIS, Paris (FR)

(21) Appl. No.: 11/870,747

(22) Filed: Oct. 11, 2007

Related U.S. Application Data
(63) Continuation of application No. 11/532,196, filed on Sep. 15, 2006, now abandoned.
Application No. 11/532,196 is a continuation of application No. PCT/FR2005/000620, filed on Mar. 15, 2005.

(30) Foreign Application Priority Data
Mar. 17, 2004 (FR) 0402824

(51) Int. Cl. A61K 31/454; A61P 11/00

(52) U.S. Cl. 514/326

(57) ABSTRACT

The present invention relates to a method for treating or preventing chronic bronchitis and chronic obstructive pulmonary disease, and also the chronic bronchitis associated with chronic pulmonary disease in a patient in need thereof, comprising administering to the patient a pharmaceutically effective amount of the pyrazole-derived compound that is an antagonist for cannabinoid CB₁ receptors.

### CROSS REFERENCE

This application is a continuation of U.S. patent application Ser. No. 11/532,196, filed Sep. 15, 2006, which is a continuation of International Patent Application No. PCT/FR2005/000620, filed Mar. 15, 2005, which claims the benefit of Priority to France application No. 0402824, filed Mar. 17, 2004.

### FIELD OF THE INVENTION

The present invention relates to the use of a pyrazole-derived compound that is an antagonist for cannabinoid CB₁ receptors, for treating or preventing chronic bronchitis and chronic obstructive pulmonary disease, and also the chronic bronchitis associated with chronic pulmonary disease in a patient in need thereof, comprising administering a pharmaceutically effective amount of the pyrazole-derived compound to the patient.

### BACKGROUND OF THE INVENTION

Endogenous cannabinoids, such as anandamide, produce profound inhibition of coughing and of bronchial muscle contraction.

It has been observed that smoked marijuana shows bronchodilatory activity, and it is known practice to use cannabinoid receptor agonists to treat various pathologies, including bronchial asthma (Tashkin D P, Shapiro B J, Lee Y E, Harper E C: Effects of smoked marihuana in experimentally induced asthma, Am. Rev. Respir. Dis. 1975, 112:377-386; Tashkin D P: Bronchial effects of aerolized delta-9-tetrahydrocannabinol in healthy and asthmatic subjects, Am. Rev. Resp. Dis. 1977, 115:57-65; Vachon L., Fitzgerald M X, Solliday N H et al.: Single-dose effects of marihuana smoke; bronchial dynamics and respiratory-center sensitivity in normal subjects, N. Engl. J. Med.

The existence of cannabinoid receptors in the respiratory pathways is described in US patent application 2002/0035150. In that application, it is indicated that blocking cannabinoid CB₁ receptors with SR 141716A has no bronchomotor effects per se, but significantly increases the bronchoconstriction and the cough caused by the administration of capsaicin.

Blocking cannabinoid CB₁ receptors with SR 141716A inhibits the anti-inflammatory effect of two produced cannabinoid agonists, HU210 and WIN 55212-2, on neutrophil migration in a mouse peritonitis model (Smith S R, Denhardt G, Terminelli C: The anti-inflammatory activities of cannabinoid receptor ligands in mouse peritonitis models, Eur. J. Pharmacol. 2001, 432:107-119).

Blocking cannabinoid CB₁ receptor antagonists “Pyrazole-derived cannabinoid receptor antagonists”, (N-piperidino-5-(4-chlorophenyl)-1-(2,4-dichlorophenyl)-4-methylpyrazole-3-carboxamide known under the code name SR141716, and the international nonproprietary name of which is rimonabant, and N-piperidino-5-(4-bromophenyl)-1-(2,4-dichlorophenyl)-4-ethylpyrazole-3-carboxamide, are described respectively in European patent applications 656354 and 1150961.

Clinical studies carried out with rimonabant have shown that it reduces hunger, calorie intake, and the body weight of obese patients (G. Le Fur, 2003, 35, First European Workshop on Cannabinoid Research, Madrid, Spain, 4-5 Apr. 2003 and Drugs RD, 2002, 3 (1), 65-66).

The results of the STRATUS clinical study in nicotine addiction have shown that rimonabant facilitates giving up smoking (Annual Scientific Session Am. Coll. Cardiol., 9 Mar. 2003, New Orleans).

However, there is no disclosure that pyrazole-derived CB₁ receptor antagonists can be active at the bronchopulmonary level and can be used in the treatment or prevention of chronic bronchitis.

### SUMMARY OF THE INVENTION

The present invention relates to a method of treating or preventing chronic bronchitis or chronic obstructive pulmonary disease (COPD) in a patient in need thereof comprising administering to the patient a pharmaceutically effective amount of a pyrazole-derived compound that is an antagonist for cannabinoid CB₁ receptors.

### DETAILED DESCRIPTION OF THE INVENTION

### Embodiments

A particular embodiment of the invention is directed to the method wherein the pyrazole-derived compound is rimonabant or N-piperidino-5-(4-bromophenyl)-1-(2,4-dichlorophenyl)-4-ethylpyrazole-3-carboxamide.

A pharmaceutical composition for use according to the present invention contain an effective dose of a pyrazole-derived compound that is an antagonist for cannabinoid CB₁ receptors, and at least one pharmaceutically acceptable excipient.

Said excipients are chosen, according to the pharmaceutical form and the method of administration desired, from the usual excipients that are known to those skilled in the art.

In the pharmaceutical compositions according to the invention for oral, sublingual, subcutaneous, intramuscular, intravenous, topical, local, intratracheal, intranasal, transdermal or rectal administration, the active principle, i.e., pyrazole-derived compound that is an antagonist for cannabinoid CB₁ receptors, can be administered in unit administration form, as a mixture with conventional pharmaceutical excipients, to patient, animal or human being, for preventing or treating the disorders of the diseases above.

Suitable unit administration forms comprise the forms for oral administration, such as tablets, soft or hard gelatin capsules, powders, granules and oral solutions or suspensions, the forms for sublingual, buccal, intratracheal, intraocular or intranasal administration and for administration by inhalation, the forms for topical, transdermal, subcutaneous, intramuscular or intravenous administration, the forms for rectal administration, and implants. For topical application, the compounds according to the invention can be used in creams, gels, ointments or lotions.

### EXAMPLE 1

### Animal Model

Migration of cells in the bronchoalveolar space after activation with bacterial LPS (lipopolysaccharide).

Mice weighing 28 to 30 g are stimulated by means of an intratracheal exposure to 10 μG of LPS. 24 hours after the injection of LPS, the animals are anaesthetized with pentobarbital and a bronchoalveolar lavage is performed. The lavage fluids are recovered and are centrifuged, and the cells are then resuspended. The number of cells is counted, differentiating the eosinophil, neutrophil and mononuclear cells according to standard morphological criteria.

The intratracheal injection of LPS induces a considerable increase in the number of mononuclear cells and neutrophils in the bronchoalveolar space of the mice. The effect of treatment with rimonabant on the LPS-induced recruitment of these cells is studied.

The rimonabant is administered to the animals 1 hour before the LPS, at doses ranging from 0.3 to 30 mg/kg/i.p. The effective dose 50 (ED₅₀) that inhibits migration of the neutrophil cells by more than 80% is 2.3 (±0.3) mg/kg. Inhibition of cell migration is comparable on the mononuclear cells: ED₅₀ equal to 1.9 (±0.5) mg/kg.

This bacterial LPS-induced model is conventionally used, in particular at the bronchopulmonary level, where it produces an infiltration of neutrophil and polymorphonuclear cells into the bronchopulmonary tissues, followed by a release of mediators that bring about tissue lesions. This infiltration of neutrophils is the result of activation of the mononuclear cells (macrophages which constitute the first barrier of defence in the bronchial epithelium, and T lymphocytes) stimulated directly by the LPS, and which release mediators (chemokines) that induce extravasation of the neutrophils and attraction of the latter towards the activated mononuclear cells. This sequence of events is entirely characteristic of the pathogenesis of chronic obstructive pulmonary disease induced by cigarette smoke and atmospheric pollution (Global Strategy for the diagnosis, management, and prevention of COPD, National Heart, Lung and Blood Institute, WHO, Executive Summary of April 1998 Meeting).

The inhibitory effect of rimonabant on the migration both of mononuclear cells and of neutrophil cells at the bronchopulmonary level, after bacterial LPS-induced activation, is reason for a therapeutic interest with respect to the indications of chronic bronchitis and chronic obstructive pulmonary disease.

1. The method of treating or preventing chronic bronchitis or chronic obstructive pulmonary disease in a patient in need thereof, comprising administering to the patient a pharmaceutically effective amount of the pyrazole-derived compound that is an antagonist for cannabinoid CB₁ receptors.

2. The method according to claim 1 wherein the pyrazole-derived compound is rimonabant or N-piperidino-5-(4-bromophenyl)-1-(2,4-dichlorophenyl)-4-ethylpyrazole-3-carboxamide.

3. The method according to claim 2 wherein the pyrazole-derived compound is rimonabant.

4. The method according to claim 2 wherein the pyrazole-derived compound is N-piperidino-5-(4-bromophenyl)-1-(2,4-dichlorophenyl)-4-ethylpyrazole-3-carboxamide.
